(19) United States
(12) Patent Application Publication
(10) Pub. No.: US 2026/0199243 A1
(43) Pub. Date: Jul. 16, 2026

(54) METHOD FOR PREPARING VONOPRAZAN WITH REDUCED NITROSAMINE

(71) Applicant: Phathom Pharmaceuticals Inc., Buffalo Grove, IL (US)

(72) Inventors: Huai-Chueh Chang, Buffalo, IL (US); James Buchanan, Buffalo Grove, IL (US); Jennifer L. Stanek, Buffalo Grove, IL (US); Tom Harris, Buffalo Grove, IL (US)

(21) Appl. No.: 19/558,801

(22) Filed: Mar. 6, 2026

Related U.S. Application Data
(63) Continuation of application No. PCT/US24/47011, filed on Sep. 17, 2024.
(60) Provisional application No. 63/583,411, filed on Sep. 18, 2023.

(51) Int. Cl. A61K 9/16; A61K 9/00; A61K 9/20; A61K 9/48; A61K 31/4439

(52) U.S. Cl. CPC A61K 9/1617; A61K 9/0053; A61K 9/1694; A61K 9/2013; A61K 9/4858; A61K 31/4439

(57) ABSTRACT

In one aspect the disclosure relates to a method of preparing a vonoprazan pharmaceutical formulation using ascorbic acid where the vonoprazan pharmaceutical formulation has reduced levels of a nitrosamine drug substance related impurity (NDSRI). The disclosure is also directed to the formulation so prepared. In one aspect, the method utilizes ascorbic acid in the wet granulation step under processing conditions that expectedly encourage instability and degradation of the ascorbic acid but result instead in successful NDSRI reduction. In another aspect, a method of preparing a vonoprazan fast-dissolving oral dosage form, such as a fast-dissolving wafer or orally-disintegrating tablet, having reduced levels of NDSRI is provided.

This application claims priority of benefit to U.S. Provisional Patent Application Ser. No. 63/583,411, filed on Sep. 18, 2023, the entire contents of which are incorporated herein by reference.

### FIELD

The disclosure relates to a method used in the preparation of a pharmaceutical formulation such as vonoprazan which results in reduced levels of nitrosamine drug substance related impurities.

### BACKGROUND

Vonoprazan is a first-in-class potassium-competitive acid blocker used to treat certain acid-related diseases such as gastroesophageal reflux disease (GERD). Vonoprazan is typically administered orally using a solid formulation such as a tablet. Clinical development has been conducted with tablets comprised of vonoprazan in 10 mg and 20 mg dosages. Methods of preparing vonoprazan tablets include the step of wet granulation, as generally described in U.S. Pat. No. 9,186,411.

Mitigation of nitrosamine drug substance related impurities (NDSRIs) and other nitrosamines in pharmaceuticals, including vonoprazan, is a pressing concern given potential health risks associated with these contaminants. Nitrosamines are thought to be present in drug products for various reasons, including the presence of certain amine compounds that have a vulnerability to form nitrosamines under reaction conditions used during drug manufacture; the presence of nitrosamines in the raw materials in the first instance; and the arising of reaction conditions during the manufacturing process that are conducive to the formation of nitrosamines. Another pathway to forming nitrosamines involves various nitrites, which can be present in excipients used in pharmaceutical formulations and which can undergo reaction with other vulnerable amines to form nitrosamines during product storage.

The U.S. Food and Drug Administration (FDA) has speculated on possible strategies, based on literature reports, to reduce the risk of NDSRIs in drug products, see “Updates on Possible Mitigation Strategies to Reduce the Risk of Nitrosamine Drug Substance-Related Impurities in Drug Products, November 2021, fda.gov. One potential strategy mentioned involves ascorbic acid. The FDA Updates identifies Nanda, K K et al. “Inhibition of N-Nitrosamine Formation in Drug Products: A Model Study” J. Pharma. Sci. 110 (2021) 3773-3775, which investigated ascorbic acid, sodium ascorbate, alpha-tocopherol, caffeic acid, and ferulic acid as potential N-nitrosamine inhibitors in oral dosage tablets made with 4-pheylpiperidine hydrochloride (4-PPHCL), an amine vulnerable to forming a corresponding nitrosamine. Nanda states the tablets were spiked with 0.57, and 5.7 μmol of each inhibitor, which according to Nanda translates into 0.1 wt % and 1.0 wt % ascorbic acid in the tablet, respectively. However, Nanda is silent on how, or at what stage, and under what processing conditions, the tablets were spiked with ascorbic acid.

This is important insofar as ascorbic acid is known to be unstable in a variety of conditions. For example, it is reported that ascorbic acid is unstable in aqueous solutions, see Yin, L. et al. “Chemical Stability of Ascorbic Acid Integrated into Commercial Products: A Review on Bioactivity and Delivery Technology,” Antioxidants, 2022, 11, 153, https://doi.org/10.3390/antiox11010153. Increases in temperature are also reported as having a detrimental effect on stability of ascorbic acid, as discussed in Jutkus, R. A. L. et al “Effect of Temperature and Initial Moisture Content on the Chemical Stability and Color Change of Various Forms of Vitamin C,” Int. J. Food Prop. 2015, 18, 862-879, which reports that for ascorbic acid samples stored at 40° C., 50° C., and 60° C., such increases in temperature led to greater chemical instability over time. Oxygen, including dissolved oxygen, has also been reported to effect the degradation of ascorbic acid, as noted in Yuan J-P et al. “Degradation of Ascorbic Acid in Aqueous Solution,” J. Agric. Food Chem. 1998, 46, 5078-5082. In addition to its instability under certain environments, the use of ascorbic acid to reduce nitrosamine formation can also have problematic outcomes, see e.g. Chang, S-K, et al. “Accelerating Effect of Ascorbic Acid on N-Nitrosamine Formation and Nitrosation by Oxyhyponitrite,” Cancer Research 3871-3872 October 1979, which concludes that the addition of ascorbic acid to a commercial product containing a variety of unknown amines might slow down the formation of some nitrosamines, but in contrast, might accelerate the formation of others. See also the circumstances in Europe involving aminophenazone preparations (an antipyretic and analgesic). In “Lessons Learnt From Presence of N-Nitrosamine Impurities in Sartan Medicines,” European Medicines Agency, HMA, 23 Jun. 2020 EMA/526934/2019 (ema.europa.eu), it is reported that in 1975, the medicines regulator in the Federal Republic of Germany, Bundesgesundheitsamt, recommended a re-formulation of aminophenazone preparations by adding ascorbic acid as an anti-oxidant to prevent in vivo nitrosation and N-Nitrosodimethylamine (NDMA) formation, but that two years later, high levels of NDMA were detected in some batches of aminophenazone and the Bundesgesundheitsamt then recommended withdrawal of aminophenazone products from the market.

Thus, while certain antioxidants are suggested by the FDA for mitigating nitrosamine formation in drug products, there is no indication that the use of ascorbic acid would be successful for vonoprazan, particularly where wet granulation and fluidization steps are employed under conditions that are otherwise thought to be hostile to the use of ascorbic acid, and including where sufficient stability of ascorbic acid is needed over time periods and storage conditions typical with a commercial pharmaceutical product.

### SUMMARY

In one aspect, the disclosure is directed to a method of preparing an aqueous granulation solution for wet granulation of vonoprazan, the method comprising providing heated water having a temperature of between about 43° C. to about 50° C.; adding fumaric acid, ascorbic acid, and a binder component, such as hydroxypropyl cellulose, to the heated water under conditions of agitation to form a heated aqueous admixture, wherein the amount of ascorbic acid added is in the range of about 0.3 wt % to about 30 wt % based on the total weight of the heated aqueous admixture; and exposing the ascorbic acid in the heated aqueous admixture to the conditions of agitation for at least about 2 hours thereby generating the aqueous granulation solution. In one practice, the fumaric acid, ascorbic acid, and binder component are added to the heated water under an oxygen-containing environment.

In another aspect, the disclosure is directed to a wet granulation method of preparing a vonoprazan composition comprising introducing a mixture of vonoprazan, including a pharmaceutically acceptable salt thereof such as vonoprazan fumarate, a filler such as a sugar alcohol, e.g. mannitol, and a disintegrant material such as a microcrystalline cellulose, into a fluidized bed processor, such as a fluidized bed dryer granulator, that has been pre-heated to a temperature of about 43° C. to about 48° C.; fluidizing the mixture with hot air at a temperature of about 43° C. to about 48° C.; and spraying the aqueous granulation solution of the disclosure onto the fluidized mixture while maintaining a temperature of about 35° C. to about 48° C. to form a vonoprazan composition comprising vonoprazan and ascorbic acid.

The disclosure also pertains to a vonoprazan composition prepared by such method herein, which composition can be used to prepare a vonoprazan pharmaceutical formulation, including an oral dosage form, e.g. a tablet. In one aspect, the vonoprazan composition and the vonoprazan pharmaceutical formulation each has a final NDSRI level between at least about 0.5% to about 100% less than a vonoprazan composition and/or a vonoprazan pharmaceutical formulation not prepared by the method herein described, e.g. prepared without using ascorbic acid. The reduced level of final NDSRI is obtained in the vonoprazan pharmaceutical formulation as initially prepared and is maintained at acceptable levels after prolonged periods of storage, e.g. 6 months or more. Vonoprazan pharmaceutical formulations prepared by the method herein include an oral dosage form of same, e.g. a tablet, having a reduced level of NDSRI as compared to such formulations not prepared with ascorbic acid.

The process conditions for the method of preparing an aqueous granulation solution for wet granulation, and for the method of wet granulation method itself, the conditions taken both individually and in totality, e.g. the presence of water in the aqueous admixture, the elevated temperatures and prolonged use of same in both methods, as well as other stressors (e.g. shear stresses involved in fluidization), and optionally the presence of an oxygen-containing atmosphere, rather than being inimical to the ascorbic acid, e.g. causing its instability and degradation, as would be expected under such conditions, instead results in successful reductions in NDSRI level, including in the ultimate vonoprazan pharmaceutical formulation, e.g. a tablet.

In another practice, the disclosure relates to a fast-dissolving oral dosage formulation of vonoprazan, such as e.g. a fast-dissolving wafer or an orally disintegrating table, a liquid solution, or a suspension, with improved palatability. In this regard, vonoprazan is known to be bitter tasting and to have other negative sensory attributes which detract from its use in oral dosage formulations such as those exemplified above. This is an important consideration for patients who have difficulty swallowing, and for pediatric patients where routes of administration and that are also easy to dose can be limited. In one aspect of this practice, the disclosure is directed to a method of preparing a fast-dissolving vonoprazan oral dosage formulation, the method comprising cooling to between about 2° C. to about 15° C., a fast-dissolving vonoprazan formulation comprising water, a binder, a sugar alcohol, an anti-oxidant, vonoprazan or a pharmaceutically acceptable salt thereof, and optionally one or more of the following: a taste-masker, a sweetener, a flavoring agent; dosing the cooled fast-dissolving vonoprazan formulation into a pre-formed mold, such as a blister pack; and freeze-drying the fast-dissolving vonoprazan formulation in the pre-formed mold to form the fast-dissolving oral dosage form. In one embodiment, the freeze drying is between about −60° C. to about −80° C. and occurs in a liquid nitrogen tunnel.

The disclosure also pertains to a fast-dissolving vonoprazan formulation prepared by such method, including e.g. a fast-dissolving wafer or an orally disintegrating table, a liquid solution or a suspension, wherein the formulation has improved palatability and the final NDSRI levels aforesaid.

### DETAILED DESCRIPTION

The entire contents of U.S. Pat. No. 9,186,411 are incorporated herein by reference for any purpose.

As used herein terms such as “a,” “an,” and “the” are not intended to refer to only a single entity but include the general class of which a specific example may be used for illustration. Terms defined herein in the singular are intended to include those terms defined in the plural and vice versa.

Reference to any numerical range as used herein expressly includes each numerical value (including fractional numbers and whole numbers) encompassed by that range and including endpoints of that range. For illustrative purposes only, a reference to a range of “0.0001 to 5000” includes whole numbers such as 5000, 4999, 4998 . . . 3, 2, 1; and includes fractional numbers such as 0.00011, 0.00012 . . . 0.1, 0.2, 0.3 . . . 1.1, 1.2, 1.3 . . . 100.5, 100.6 . . . 4900.5, 4990.6, 4990.7 etc.

As used herein, the term “about” includes the value listed and indicates that the value listed may be somewhat altered, as long as the alteration does not result in nonconformance of the article or method or system herein described. For example, the term “about” as used herein can refer to a variation of between ±1% up to ±10%, including any value therebetween.

As used herein the phrase “gastroesophageal reflux disease” or “GERD” includes but is not limited to disorders and/or conditions wherein stomach acid repeatedly flows back into the esophagus and which can irritate the esophageal lining. Symptoms include, without limitation, burning sensation in the chest, regurgitation of food or sour liquid, upper abdominal pain, dysphagia, coughing, vocal cord inflammation, new or worsened asthma.

As used herein the term “subject” refers to any animal, including mammals. Exemplary subjects include, but are not limited to, mice, rats, rabbits, dogs, cats, swine, cattle, sheep, horses, primates, and humans. In one embodiment, the subject is a human.

As used herein, the term “treating” or “treatment” refers to reversing, alleviating, reducing, and/or inhibiting the progress of gastroesophageal reflux disease (GERD), for example, reversing, alleviating, reducing and/or inhibiting any of the symptoms of gastroesophageal reflux disease (GERD) in an individual who is experiencing or displaying same, such as without limitation, burning sensation in the chest, regurgitation of food or sour liquid, upper abdominal pain, dysphagia, coughing, vocal cord inflammation, new or worsened asthma.

As used herein, the phrase “therapeutically effective amount” refers to the amount of a vonoprazan formulation prepared according to the method of the disclosure that elicits the biological or medicinal response that is being sought in a subject such as a human by a medical doctor or other clinician, i.e. relieving the symptoms of gastroesophageal reflux disease (GERD) as described above. Those in the art understand that the therapeutically effective amount can vary with the particulars of the subject to whom the formulation is being administered, e.g. age, weight, other health or metabolic factors, which can affect bioavailability and plasma concentrations which can influence what is a therapeutically effective amount, all within the ranges disclosed herein.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002. In one practice, the salt for vonoprazan is fumarate.

Generally, the process for preparing a vonoprazan pharmaceutical formulation, such as a tablet, entails preparing an aqueous granulation solution comprising water, a binder component such as a crystalline cellulose e.g. hydroxypropyl cellulose, and fumaric acid; and preparing a bowl charge comprising vonoprazan, a filler such as a milled sugar alcohol, e.g. mannitol, and a disintegrant material, e.g. microcrystalline cellulose. The aqueous granulation solution and bowl charge are introduced into a fluidized bed processor, such as a fluidized bed dryer granulator, for granulation which produces a vonoprazan composition in the form of a granulated powder. The granulated powder thus formed is milled, whereafter blending of additional raw materials such as excipients, e.g. lubricants, is performed. After blending, the product that results is compressed to form tablets, including tablets comprising 10 mg and 20 mg vonoprazan or a pharmaceutically acceptable salt thereof, e.g. fumarate.

### The Aqueous Granulation Solution and Method:

In one embodiment, the disclosure is directed to a method of preparing an aqueous granulation solution which is useful in the wet granulation of vonoprazan. In one practice, the method comprises the steps of providing heated water having a temperature of between about 43º° C. to about 50° C., including about 45° C. to about 50° C., and about 48° C. to about 50° C. which water can be heated by means known in the art, e.g. by having the pertaining mixing vessel jacketed or otherwise supplied with a heat source. The method further comprises adding fumaric acid, ascorbic acid, and a binder component to the heated water under conditions of agitation to form a heated aqueous admixture. The binder component includes those known in the art, e.g. cellulosic materials such as hydroxypropyl cellulose, and includes use of an individual binder component or mixtures of binder components. In one practice, the mixing vessel is a metal mixing vessel comprising a stainless steel or other suitable alloy or material of construction.

The mixing vessel can comprise agitation means as known in the art, e.g. a mixer configuration comprising at least one impeller having a least one blade per impeller, e.g. an impeller have 3 to 6 blades, such as commercially available and known as a Lightnin mixer. In one practice, the at least one rotating impeller blade causes aeration of the heated aqueous mixture. Operationally, the conditions of agitation can comprise constant agitation, intermittent agitation, or variable agitation. In one practice, the conditions of agitation comprise an impeller speed of between about 10 rpm to about 1500 rpm. In one practice, the mixer configuration and/or operational conditions results in aeration of the heated aqueous admixture, which aeration can provide or augment any presence of oxygen, e.g. if an oxygen-containing atmosphere or other oxygen source is present. The method comprises the step of exposing the ascorbic acid in the heated aqueous admixture to the conditions of agitation for at least about 2 hours thereby generating the aqueous granulation solution.

In one practice for forming the heated aqueous admixture, the amount of ascorbic acid added is in the range of between about 0.3 wt % to about 30 wt % based on the total weight of the heated aqueous admixture; in other practices, the amount of ascorbic acid added is in the ranges of about 0.3 wt % to about 10 wt %; about 0.3 wt % to about 5.0 wt %; about 0.3% to about 3%; about 0.3 wt % to about 1.5 wt %; about 0.5 wt % to about 1.5 wt %, about 2 wt % to about 4 wt % (e.g. about 2.6 wt % to about 3.2 wt %). In another practice, the amount of ascorbic acid added is about 3.0 wt % based on the total weight of the heated aqueous admixture. Ascorbic acid can be provided as a liquid or solid, e.g. a powder. In one practice, the fumaric acid is present at about 0.1 wt % to about 0.3 wt %, including about 0.1 wt % to about 0.2 wt %; about 0.15 wt % to about 0.25 wt %; about 0.17 wt % to about 0.21 wt %; and the binder component is present at about 5.0 wt % to about 7.0 wt %; including about 5.2 wt % to about 6.4 wt % (e.g. about 5.24 wt %) of the heated aqueous admixture. The sequence of addition of the fumaric acid, ascorbic acid, and the binder component to the heated water can vary and in one practice, the sequence of addition is fumaric acid, ascorbic acid, and then the binder component. Mixing times after the addition of fumaric acid and after the addition of ascorbic acid can vary, and in one practice the mixing time is up to about 20 minutes, e.g. up to 10 minutes, until all material is dissolved. In this practice, where the binder component is added last, the mixing time after adding the binder component can vary and be up to about 2 hours, e.g. about 1.5 hours, and the ascorbic acid in the heated aqueous admixture is exposed to the agitation for at least about 2 hours. In one practice, the heated aqueous admixture has a pH of between about 2.0 to about 3.0. In one optional embodiment, the fumaric acid, the ascorbic acid, and the binder component are added to the heated water under an oxygen-containing atmosphere, and the ascorbic acid in the heated aqueous mixture is exposed to the oxygen-containing atmosphere and the conditions of agitation for at least about 2 hours thereby generating the aqueous granulation solution. The oxygen-containing environment can comprise exposing admixture to environmental air and/or air in the head space of metal mixing vessel; in such circumstances, the exposure can be augmented by aeration created by the agitation.

In one practice for the aqueous granulation solution, the water is present at about 90 wt % to about 92 wt %; the fumaric acid is present at about 0.1 wt % to about 0.2 wt %; the binder component is present is present at about 5.2 wt % to about 6.4 wt %; and the ascorbic acid is present at about 2.6 wt % to about 3.2 wt %, all as based on the total weight of the aqueous granulation solution. The aqueous granulation solution produced by the method herein is stable for a storage time of up to about 28 hours. In various embodiments in this regard, the amount of ascorbic acid in the aqueous granulation solution present during the storage time is no less than about 80%, no less than about 85%, no less than about 90%, no less than about 95% of the amount of ascorbic acid that was added to the heated aqueous admixture.

The disclosure is also directed to the aqueous granulation solution prepared by the method described herein. In another embodiment, the disclosure is directed to an aqueous granulation solution comprising water, fumaric acid, a binder component, and ascorbic acid. In one aspect the water is present at about 90 wt % to about 92 wt %; the fumaric acid is present at about 0.17 wt % to about 0.21 wt %; the binder component is hydroxypropyl cellulose present at about 5.24 wt % to about 6.41 wt %; and the ascorbic acid is present at about 2.62 wt % to about 3.2 wt %, all as based on the total weight of the aqueous granulation solution.

### The Vonoprazan Composition and Method:

In another embodiment, the disclosure is directed to a wet granulation method for preparing a vonoprazan composition, which composition can be used to prepare a vonoprazan pharmaceutical formulation, the method comprising introducing a mixture of vonoprazan, e.g. a vonoprazan pharmaceutically acceptable salt such as vonoprazan fumarate, a filler such as a sugar alcohol, e.g. mannitol, including milled mannitol, and a disintegrant material such as a crystalline cellulose, e.g. microcrystalline cellulose, into a fluidized bed processor pre-heated to a temperature of about 43° C. to about 48° C. In one instance, the mixture comprises about 11% wt % to about 15 wt % vonoprazan, including about 11.6 wt % to about 14.2 wt % vonoprazan; about 62 wt % to about 80 wt % filler, including about 64.4 wt % to about 78.7 wt % filler, e.g. milled mannitol; and about 9 wt % to about 12 wt %, including about 9.6 wt % to about 11.7 wt % disintegrant material, e.g. microcrystalline cellulose, based on the total weight of the mixture. The fluidized bed processor can comprise those known in the art, e.g. a fluidized bed dryer granulator such as commercially available from Glatt, including the GPCG Pro 300, GPCG-60 and the like. The vonoprazan composition produced is in the form of a granulated powder. In one practice, the method further comprises the steps of fluidizing the mixture with hot air at a temperature of about 43° C. to about 48° C.; and spraying the aqueous granulation solution prepared as described herein onto the fluidized mixture while maintaining a temperature of about 35° C. to about 48° C., including from about 35° C. to about 43° C., from about 35° C. to about 38° C., and from about 43° C. to about 48° C., to form a vonoprazan composition comprising vonoprazan, e.g. in the form of vonoprazan fumarate, and ascorbic acid. The vonoprazan composition produced is in the form of a granulated powder. In one practice, the amount of ascorbic acid in the vonoprazan composition is about 1.4 wt % to about 1.8 wt % based on the total weight of the vonoprazan composition. The disclosure is also directed to a vonoprazan composition prepared by the method described herein.

In one practice, the vonoprazan composition thus formed is used to prepare a vonoprazan pharmaceutical formulation for administration to a patient. As a non-limiting example in this regard, the method further comprises blending the vonoprazan composition thus formed with a lubricant as known in the art, e.g. one or more stearates such as magnesium stearate, calcium stearate, zinc stearate, sodium stearyl fumarate to form a mixed vonoprazan composition, which can then be formed into a tablet, by means known in the art, comprising the mixed vonoprazan composition. In various embodiments the tablet comprises (i) about 13.0 mg to about 14.0 mg of vonoprazan fumarate, including about 13.36 m of vonoprazan fumarate; and about 1 mg to about 2 mg of ascorbic acid, including about 1.4 mg to about 1.65 mg ascorbic acid; or (ii) about 26.0 mg to about 27.0 mg of vonoprazan fumarate, including about 26.72 mg of vonoprazan fumarate; and about 2.5 to about 3.5 mg of ascorbic acid, including about 2.8 mg to about 3.3 mg ascorbic acid.

In one practice, the vonoprazan composition derived from wet granulation disclosed herein and the vonoprazan pharmaceutical formulation, e.g. a tablet, as prepared by the method disclosed using ascorbic acid each has a final NDSRI level between at least about 0.5% to 100%, including between at least about 1% to about 99.9%, including between about 10% to about 95%, between about 20% and 90%, and between about 30% and about 85% less, and about 40% to about 80% less than a vonoprazan composition and/or pharmaceutical formulation not prepared by the method disclosed herein, e.g. prepared without using ascorbic acid in the method otherwise disclosed herein. The percentage ranges herein include all integers and fractional percentage values in between and inclusive of range end points, e.g. 5%, 6%, 7% . . . 10%, 20%, 30%, 40%, 50%, 60%, 70%, 80%, 90%, 95% etc.; and includes circumstances where no NDSRI is detected after practice of the method.

Methods to measure the level of NDSRI used to determine the percentage difference in levels of NDSRI include those known in the art, e.g. liquid chromatography-high resolution mass spectrometry (LC-HRMS). In one practice, the final level of NDSRI in a vonoprazan composition and/or vonoprazan pharmaceutical formulation not prepared by the method disclosed herein, e.g. prepared without using ascorbic acid, can range from about 0.5 ppm to about 1.5 ppm or higher, whereas in the practices of the disclosure, the final level of NDSRI in the vonoprazan composition and the vonoprazan pharmaceutical formulation e.g., a tablet, prepared using ascorbic acid after practice of the method herein ranges from levels of NDSRI that are 0%, or are undetectable, and can be up to about 0.60 ppm, including up to about 0.20 ppm, e.g. from about 0.10 ppm to about 0.20 ppm. In other practices, the levels of NDSRI provided by the method disclosed translate into a reduction in NDSRI level of about 60% to about 93% or more, e.g. about 98%. In various embodiments, a tablet prepared by the method of the disclosure has an NDSRI level of less than about 0.60 ppm; less than about 0.20 ppm; and less than about 0.10 ppm. The reduced final levels of NDSRI obtained by the method herein applies to the vonoprazan pharmaceutical formulation as initially prepared; after prolonged periods of storage, e.g. up to 6 months or longer, the final levels of NDSRI do not exceed 2.4 ppm.

The disclosure is also directed to a vonoprazan pharmaceutical formulation comprising (i) about 13 mg to about 14 mg (e.g. about 13.36 mg) of vonoprazan fumarate, and about 1 mg to about 2 mg ascorbic acid, including about 1.4 mg to about 1.65 mg ascorbic acid; or (ii) about 26 mg to about 27 mg (e.g. about 26.72 mg) mg of vonoprazan fumarate, and about 2.5 mg to about 3.5 mg ascorbic acid, including about 2.8 mg to about 3.3 mg ascorbic acid. In various practices, the vonoprazan pharmaceutical formulation comprises a tablet or a capsule. The vonoprazan pharmaceutical formulation of the disclosure has an NDSRI level of less than about 0.10 ppm; and less than about 0.06 ppm.

The vonoprazan pharmaceutical formulation prepared by the method herein may be administered alone or in combination with one or more other drugs (or as any combination thereof). Administration can be oral.

Serviceable oral formulations include solid formulations e.g. tablets, pills, powders, lozenges (including liquid filled), caplets, or capsules (containing particulates, liquids, or powders), chewables, multi- and nano-particulates, gels, solid solutions, liposomes, films (including muco-adhesive), ovules, sprays and liquid formulations. Liquid formulations include suspensions, solutions, syrups, and elixirs. Such formulations can be employed in soft or hard capsules and can comprise a carrier such as water, ethanol, polyethylene glycol, methylcellulose, or a suitable oil; and one or more emulsifying agents and/or suspending agents. Liquid formulations may also be prepared by the reconstitution of a solid, e.g. from a sachet.

In tablet dosage forms, depending upon the dose, vonoprazan including pharmaceutically acceptable salts thereof may representatively make up from 0.5% weight to 95% weight of the tablet, e.g. from 5% to about 60% weight of the tablet. Tablets can additionally comprise one or more disintegrants as known in the art, e.g. microcrystalline cellulose, sodium starch glycolate, sodium carboxymethyl cellulose, calcium carboxymethyl cellulose, alkyl-substituted hydroxypropyl cellulose, starch, pre-gelatinized starch and sodium alginate. Typically, the disintegrant can comprise from 1% weight to 25% weight of the tablet. Tablets can also comprise one or more binders to impart cohesiveness to the formulation. Serviceable binders as known in the art include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also comprise one or more diluents as known in the art, e.g. lactose (monohydrate, spray-dried monohydrate, anhydrous and the like), mannitol, xylitol, dextrose, sorbitol, microcrystalline cellulose, starch and dibasic calcium phosphate dehydrate. Tablets may also comprise one or more active agents as known in the art, e.g. sodium lauryl sulfate and polysorbate 80, and one or more glidants, e.g. silicon dioxide and talc. Surface active agents, if used, typically comprise from 0.2% weight to 5% weight of the tablet; glidants can comprise from 0.2% weight to 1% weight of the tablet. Tablets can also comprise one or more lubricants as known in the art, e.g. magnesium stearate, calcium stearate, zinc stearate, sodium stearyl fumarate. Lubricants typically comprise from 0.25% weight to 10% weight, e.g. from 0.5% weight to 3% weight of the tablet. The tablet may comprise other ingredients such as anti-oxidants, colorants, flavoring agents, preservatives and taste-masking agents. Without limitation, a representative tablet comprises up to about 80% of the vonoprazan composition from about 10% weight to about 90% binder, from about 0% weight to about 85% weight diluents, from about 2% weight to about 10% weight disintegrant, and from about 0.25% weight to about 10% weight lubricant. The actual tablet form may be produced by methods known in the art, e.g. from tablet blends that may be compressed directly or by roller to form tablets. The final tablet may comprise one or more layers and may be coated or uncoated.

In another embodiment the disclosure is directed to a method of treating gastroesophageal reflux disease (GERD) comprising administering to a patient in need thereof a therapeutically effective amount of the vonoprazan pharmaceutical formulation, e.g. a tablet, that comprises ascorbic acid. In one practice, the amount of ascorbic acid is greater than zero and up to about 4.0 mg, e.g. between about 1.4 mg to about 3.3 mg. In one aspect, the vonoprazan pharmaceutical formulation comprises a tablet of about 10 mg vonoprazan or pharmaceutically acceptable salt thereof, such as fumarate, e.g. vonoprazan fumarate, and up to about 2.0 mg of ascorbic acid, e.g. about 1.4 mg to about 1.65 mg ascorbic acid. In another aspect, the tablet comprises about 20 mg of vonoprazan or pharmaceutically acceptable salt thereof, such as fumarate, e.g. vonoprazan fumarate, and up to about 3.0 mg ascorbic acid, e.g. about 2.8 mg to about 3.3 mg ascorbic acid. In one practice, the vonoprazan pharmaceutical formulation comprising ascorbic acid is prepared by the method described herein.

In another embodiment, the disclosure is directed to a package comprising a pharmaceutical composition comprising a vonoprazan pharmaceutical formulation, prepared according to the method herein, in unit doses for treatment of gastroesophageal reflux disease (GERD). In one practice, the package comprises instructions, e.g. on a written insert, for performing the treatment in a therapeutically effective manner. In one embodiment, the vonoprazan pharmaceutical formulation is for oral administration, e.g. is in the form of a tablet. In one embodiment, the package comprises multiple vonoprazan pharmaceutical formulations which may have the same or different doses. In some embodiments, the package comprises one or more containers, blister packs, or other forms of pharmaceutical packaging. In various embodiments, the vonoprazan pharmaceutical formulations is in tablet form comprising (i) about 13 mg to about 14 mg (e.g. about 13.36 mg) of vonoprazan fumarate, and about 1 mg to about 2 mg ascorbic acid, including about 1.4 mg to about 1.65 mg ascorbic acid; or (ii) about 26 mg to about 27 mg (e.g. about 26.72 mg) of vonoprazan fumarate, and about 2.5 mg to about 3.5 mg ascorbic acid, including about 2.8 mg to about 3.3 mg ascorbic acid.

### EXAMPLES

The use of water in the aqueous admixture, the high temperature of the heated aqueous admixture, and the optional exposure to an oxygen-containing environment to form the aqueous granulation solution as described herein are all thought to be inimical to ascorbic acid and to prefigure instability and degradation of same rather than lead to successful reductions in levels of NDSRI.

### Example 1: Aqueous Granulation Solution Comprising Ascorbic Acid

An aqueous granulation solution was prepared according to the method herein described by heating purified water (51.6 kg) to 45° C. (range for this example: 43° C. to 48° C.) using a jacketed kettle. Fumaric acid (0.1 kg) was then added to the heated water and mixed until completely dissolved (10 minutes at a mixer speed of 1065 rpm). Ascorbic acid (1.650 kg) was added to the solution and mixed until visually dissolved (mixed for 20 minutes at a mixer speed of 1065 rpm). Finally, hydroxypropyl cellulose (3.300 kg) was added to the solution and mixed until completely dissolved (mixed for 100 minutes at a mixer speed of 1063 rpm) to form the aqueous granulation solution. The aqueous granulation solution was prepared with 10% excess to account for priming the pumps and tubing.

### Example 2: Wet Granulation

In accordance with the wet granulation method described herein, a fluid bed dryer granulator (GPCG-60), was pre-conditioned for 10 minutes at 42° C. to 46° C. At steady-state, vonoprazan fumarate (12.16 kg), mannitol (67.12 kg) and crystalline cellulose (10.0 kg) were placed in the fluidized bed dryer granulator and the mixture was preheated to a range of 42° C. to 46° C. to a product temperature of 44° C. The mixture was wet granulated by spraying an aqueous binder solution (51.51 kg) prepared in accordance with Example 1 but comprising the following amounts of hydroxypropylcellulose (3.0 kg), fumaric acid (0.1 kg) and ascorbic acid (1.5 kg), to form of a granulated powder. The conditions were maintained at 35° C. (range 33° C. to 37° C.) throughout the spray phase. The obtained vonoprazan composition in the form of a granulated powder (90.84 kg) was passed through a powermill (Comil equipped with a 2F055 round screen) to give a sized powder. The sized powder (90.84 kg), croscarmellose sodium (4.83 kg) and magnesium stearate (0.97 g) were placed in a V-blender mixer, and mixed to give a mixed powder. A vonoprazan pharmaceutical formulation in the form of a tablet was prepared by having the mixed powder tableted by a rotary tableting machine.

### Example 3: NDSRI Levels

### Vonoprazan Tablets Prepared Without Ascorbic Acid:

Bulk lots tested in all examples herein were prepared at Phathom Pharmaceuticals' manufacturing site.

A bulk lot of vonoprazan tablets (20 mg) were prepared by a conventional method without ascorbic acid. Initial NDSRI levels for tablets in this lot (measured 4 months after manufacture) ranged from 1.18 ppm to 1.42 ppm. NDSRI levels for tablets in this lot after 1 month, 2 months, 3 months, and 6 months of storage at 25° C. and 60% relative humidity (RH), ranged from 1.46 to 1.97. NDSRI levels for tablets in this lot after 1 month, 2 months, 3 months, and 6 months of storage at 40° C. and 75% RH ranged from 1.84 ppm to 2.50 ppm.

Additional bulk lots of vonoprazan tablets (20 mg) prepared similarly had NDSRI levels that ranged from 1.14 ppm to 2.83 ppm after 3 months of storage at 25° C. and 60% RH; and that ranged from 1.31 ppm to 3.18 ppm after 6 months of storage at 25° C. and 60% RH; and that ranged from 1.31 ppm to 3.33 ppm after 3 months of storage at 40° C. and 75% RH, and that ranged from 1.52 ppm to 3.68 ppm after 6 months of storage at 40° C. and 75% RH.

### Vonoprazan Tablets Prepared by the Method of the Disclosure:

Bulk lots of vonoprazan tablets (10 mg and 20 mg) comprising 1.5 wt % ascorbic acid were prepared in accordance with the process parameters of Example 2. Initial NDSRI levels for tablets in these lots (measured within 1 month after manufacture) ranged from below the Level of Quantification (LOQ), i.e. less than 0.10 ppm, to 0.18 ppm. Test Results for N-Nitroso-Vonoprazan levels (NDSRI) levels in ppm for tablets made in these bulk lots packaged in 10 mg 30-ct Bottles, 20 mg 5-ct Bottles, and 20 mg ct-Bottles were measured for Long-Term storage at 25° C. and 60% relative humidity (RH) and for Accelerated Storage at 40° C. and 75% RH after a periods of 12 months and 6 months, respectively. The results are shown in the Table below:

Stability Test Results for N-Nitroso-Vonoprazan (ppm)

| | | Long-Term 25° C./60% RH | | | | | | Accelerated 40° C./75% RH | | | |
|---|---|---|---|---|---|---|---|---|---|---|---|
| Lot | Initial | 1 mo | 2 mo | 3 mo | 6 mo | 9 mo | 12 mo | 1 mo | 2 mo | 3 mo | 6 mo |

10 mg 5-ct Bottles

| | | | | | | | | | | |
|---|---|---|---|---|---|---|---|---|---|---|
| 0.11 | 0.11 | — | 0.14 | <LOQ | 0.10 | 0.10 | 0.11 | — | 0.19 | 0.13 |
| 0.19 | 0.19 | — | 0.19 | 0.22 | 0.20 | 0.20 | 0.20 | — | 0.22 | 0.21 |
| 0.11 | 0.11 | — | 0.15 | 0.10 | 0.12 | 0.11 | 0.12 | — | 0.16 | 0.13 |

10 mg 30-ct Bottles

| | | | | | | | | | | |
|---|---|---|---|---|---|---|---|---|---|---|
| 0.11 | <LOQ | 0.17 | <LOQ | 0.10 | <LOQ | 0.33 | 0.11 | 0.23 | 0.27 | <LOQ |
| 0.18 | 0.11 | 0.24 | 0.17 | 0.25 | 0.17 | 0.41 | 0.19 | 0.32 | 0.36 | 0.16 |
| <LOQ | 0.14 | 0.14 | 0.16 | 0.11 | <LOQ | <LOQ | 0.17 | 0.17 | 0.24 | 0.10 |

20 mg 5-ct Bottles

| | | | | | | | | | | |
|---|---|---|---|---|---|---|---|---|---|---|
| 0.13 | 0.17 | 0.19 | 0.16 | 0.13 | 0.13 | 0.34 | 0.21 | 0.31 | 0.23 | 0.14 |
| <LOQ | 0.13 | 0.15 | 0.12 | 0.11 | <LOQ | 0.29 | 0.16 | 0.23 | 0.22 | 0.14 |
| <LOQ | 0.15 | 0.16 | 0.18 | 0.12 | 0.10 | <LOQ | 0.19 | 0.23 | 0.26 | 0.13 |

20 mg 30-ct Bottles

| | | | | | | | | | | |
|---|---|---|---|---|---|---|---|---|---|---|
| 0.13 | <LOQ | 0.18 | 0.16 | 0.12 | 0.12 | 0.33 | 0.12 | 0.24 | 0.27 | 0.15 |
| <LOQ | <LOQ | 0.14 | 0.13 | <LOQ | <LOQ | 0.29 | <LOQ | 0.19 | 0.23 | <LOQ |
| <LOQ | 0.16 | 0.16 | 0.18 | 0.12 | 0.10 | <LOQ | 0.17 | 0.19 | 0.24 | 0.14 |

20 mg Blisters

| | | | | | | | | | | |
|---|---|---|---|---|---|---|---|---|---|---|
| 0.16 | 0.14 | 0.14 | 0.14 | 0.11 | 0.14 | 0.13 | 0.13 | 0.15 | 0.21 | 0.11 |
| 0.13 | 0.10 | 0.11 | 0.12 | <LOQ | <LOQ | <LOQ | 0.10 | 0.13 | 0.19 | <LOQ |
| 0.18 | 0.14 | 0.15 | 0.16 | 0.10 | 0.15 | 0.14 | 0.14 | 0.17 | 0.25 | 0.11 |

— = No Test scheduled;
LOQ = Limit of Quantitation (0.10 ppm)

In another aspect, the disclosure relates to a method for the preparation of a vonoprazan pharmaceutical formulation and the pharmaceutical formulation thus made using ascorbic acid as described herein where the ascorbic acid is used in combination with any one or more of the following compounds: cysteine (e.g. cysteine HCl), tartaric acid, gallic acid, sodium ascorbate, α-tocopherol, maltol, resveratrol, propyl gallate, beta hydroxyl acids (BHA, e.g. salicylic acid), butylated hydroxytoluene (BHT), caffeic acid, ferulic acid, α-amino acids (e.g. glycine, lysine, histidine), L-cystine, p-aminobenzoic acid (PABA), urea, sodium sulfate, ammonium chloride, sodium carbonate. In another aspect, the disclosure relates to a method for the preparation of a vonoprazan pharmaceutical formulation and the pharmaceutical formulation thus made using any one or more of the foregoing compounds in lieu of ascorbic acid under the conditions disclosed herein. The pharmaceutical formulation thus made are useful in treating GERD as disclosed herein.

### Fast-Dissolving Vonoprazan Formulation and Method

In one practice, a method of lyophilization is used to prepare a fast-dissolving vonoprazan oral dosage formulation. Broadly, in one non-limiting practice, the method comprises dissolving or dispersing the vonoprazan of a pharmaceutically acceptable salt thereof, in an aqueous solution with an anti-oxidant and excipients such as a binder, e.g. gelatin and/or a sugar alcohol, e.g. mannitol, which are used as carrier or matrix materials. Additional ingredients such as sweeteners, e.g. sucralose, and flavoring agents as known in the art and/or taste-masking agents such as ion-exchange resins or cyclodextrins, e.g. hydroxypropyl-β-cyclodextrin, may be optionally incorporated. In this general methodology, the resulting mixture, which is dosed in a pre-formed mold, e.g. a pre-formed blister pack, is rapidly frozen, e.g., in a liquid nitrogen tunnel at e.g. about −70° C. directly in the pockets of pre-formed blister packs to remove the water, followed by a separate subsequent freeze-drying process. Once the product is dried, the blisters are immediately sealed. This type of preparation process at low temperatures combined with minimal exposure to air and water and in the absence of excipients (e.g. magnesium stearate) known to contribute to nitrosamine formation collectively minimize the potential formation of NDSRI.

Methods of preparation a fast-dissolving vonoprazan oral dosage formulation as described herein are exemplary. The sequence of steps in which the ingredients are provided or combined are not limited to the descriptions herein and can be varied. In one such method of preparation, a binder and a sugar alcohol as previously defined, are combined with water and stirred at a temperature of about 60° C.±2° C. for about 60 minutes to form a pre-mix. Suitable binders and sugar alcohols are as previously defined and include e.g. fish gelatin such as high molecular weight (HMW) fish gelatin as a binder; and mannitol as a sugar alcohol. The pre-mix is cooled, e.g. to 30° C.±2° C. and optionally filtered, e.g. through a 35-40 μm sieve. The pre-mix is then further cooled and maintained at a temperature of 10° C.±2° C. Separately, vonoprazan fumarate is combined with an anti-oxidant and optionally a taste-masker. Suitable anti-oxidants include, without limitation, ascorbic acid, vitamin E, cysteine, methionine, uric acid, tartaric acid, gallic acid, sodium metabisulfite, and combinations of same. Suitable taste-maskers include those in the art such as, without limitation, ion-exchange resins and cyclodextrins including e.g. hydroxypropyl β-cyclodextrin.

In one particular embodiment, the method of preparing a fast-dissolving vonoprazan oral dosage formulation comprises the steps of (i) cooling to between about 2° C. to about 15° C., a fast-dissolving vonoprazan formulation comprising water, a binder, a sugar alcohol, an anti-oxidant, vonoprazan or a pharmaceutically acceptable salt thereof, and optionally one or more of the following: a taste-masker, a sweetener, a flavoring agent. The water, binder, sugar alcohol, anti-oxidant, vonoprazan or a pharmaceutically acceptable salt thereof, and the taste-masker, sweetener, and flavoring agent to the extent any of these optional ingredients are present, can be combined in any sequence; in one practice, the binder and sugar alcohol are first combined to form a pre-mix with the anti-oxidant, vonoprazan or a pharmaceutically acceptable salt thereof, and any of the taste-masker, sweetener, and flavoring agent being added thereafter. The next step (ii) in the method of this embodiment is dosing the cooled fast-dissolving vonoprazan formulation thus cooled into a pre-formed mold, such as a blister pack. Step (iii) of this embodiment entails freeze-drying the fast-dissolving vonoprazan formulation that is in the pre-formed mold to form the fast-dissolving oral dosage form. The freeze drying can performed by techniques known in the art, including in one non-limiting practice freeze drying at between about −50° C. to −80° C., e.g. about −70° C.; this freeze drying can occur in a liquid nitrogen tunnel.

The binder comprises those as defined herein, e.g. a gelatin such as a fish gelatin, e.g. high molecular weight (HMW) fish gelatin; mixtures of binders can be used. The sugar alcohol comprises those as defined herein, e.g. mannitol; mixtures of sugar alcohols can be used. The anti-oxidant comprises ascorbic acid, vitamin E, cysteine (e.g. cysteine HCl), methionine, uric acid, tartaric acid, gallic acid, sodium metabisulfite; mixtures of any of the foregoing can be used. The taste-masker comprises those in the art, such as without limitation a cyclodextrin, e.g. a hydroxypropyl β-cyclodextrin; mixtures of taste-maskers can be used. The pharmaceutically acceptable salts of vonoprazan include those as defined herein, e.g. vonoprazan fumarate; mixtures of pharmaceutically acceptable salts can be used. The sweetener comprises those known in the art, such as without limitation, sucralose; mixtures of sweeteners can be used. The flavoring agent comprises those known in the art, such as without limitation, flavoring agents that are orange flavored, fruit punch flavored, tropical flavored, wild berry flavored; mixtures of flavoring agents can be used.

In a first embodiment, the fast-dissolving vonoprazan formulation comprises the following, as based on the total weight of the fast-dissolving vonoprazan formulation: the vonoprazan fumarate is present at about 1 wt % to about 4 wt %; in other practices the vonoprazan fumarate is present at about 1 wt % to about 2 wt %; and about 1.5 wt %. The water is present at about 70 wt % to about 90 wt %; at about 80 wt % to about 85 wt %; and about 82 wt % to about 83 wt %. The binder is present at about 3 wt % to about 6 wt %; about 4 wt % to about 5 wt %; and about 4.5 wt %. The sugar alcohol is present at about 2 wt % to about 6 wt %; about 2 wt % to about 4 wt %; and about 3.6 wt %. The taste-masker is present at about 3 wt % to about 10 wt %; about 6 wt % to about 7 wt %; and about 6.75 wt %. The anti-oxidant is present at about 0.1 wt % to about 1 wt %; about 0.3 wt % to about 0.7 wt %; and about 0.5 wt %. The sweetener is present at about 0.1 wt % to about 0.5 wt %; about 0.2 wt % to about 0.3 wt %; and about 0.25 wt %. The flavoring agent is present at about 0.1 wt % to about 1 wt %; about 0.3 wt % to about 0.5 wt %; and about 0.4 wt %.

In one practice of this first embodiment the fast-dissolving vonoprazan formulation, the binder comprises a fish gelatin, the sugar alcohol comprises mannitol, the vonoprazan comprises vonoprazan fumarate, the anti-oxidant comprises cysteine, the taste-masker is present and comprises hydroxypropyl β-cyclodextrin, the sweetener is present and comprises sucralose. In one aspect of this practice, the flavoring agent is present and is orange flavored, fruit punch flavored, tropical flavored, wild berry flavored, or combinations of the foregoing.

In a second embodiment, the fast-dissolving vonoprazan formulation comprises the following, as based on the total weight of the fast-dissolving vonoprazan formulation: the vonoprazan fumarate is present at about 3 wt % to about 6 wt %; in other practices the vonoprazan fumarate is present at about 4 wt % to about 5 wt %; and about 4.45 wt %. The water is present at about 70 wt % to about 90 wt %; about 80 wt % to about 90 wt %; and about 85 wt %. The binder is present at about 3 wt % to about 7 wt %; about 4 wt % to about 6 wt %; and about 5 wt %. The sugar alcohol is present at about 2 wt % to about 6 wt %; about 3 wt % to about 5 wt %; and about 4 wt %. The taste-masker is absent. The anti-oxidant is present at about 0.1 wt % to about 1 wt %; about 0.3 wt % to about 0.7 wt %; and about 0.5 wt %. The sweetener is present at about 0.1 wt % to about 1 wt %; about 0.2 wt % to about 0.8 wt %; and about 0.5 wt %. The flavoring agent is present at about 0.1 wt % to about 1 wt %; about 0.3 wt % to about 0.5 wt %; and about 0.4 wt %.

In one practice of this second embodiment the fast-dissolving vonoprazan formulation, the binder is fish gelatin; the sugar alcohol is mannitol; the anti-oxidant is tartaric acid; and the sweetener is sucralose. In one aspect of this practice, the flavoring agent is present and is orange flavored, fruit punch flavored, tropical flavored, wild berry flavored, or combinations of the foregoing.

The fast-dissolving oral dosage form prepared by the method comprises a vonoprazan wafer, a vonoprazan orally disintegrating tablet, a vonoprazan liquid, or a vonoprazan suspension. The fast-dissolving oral dosage form prepared by the method, such as the vonoprazan wafer or vonoprazan orally disintegrating tablet, has a final NDSRI level, as measured herein, between at least about 0.5% to 100% less than a comparison vonoprazan wafer or vonoprazan orally disintegrating tablet prepared without the anti-oxidant.

In another aspect, the disclosure is directed to a method of treating gastroesophageal reflux disease (GERD) comprising administering to a patient in need thereof a therapeutically effective amount of the fast-dissolving vonoprazan formulation, e.g. produced by the described herein, and comprising vonoprazan or a pharmaceutically acceptable salt thereof, water, a binder, a sugar alcohol, an anti-oxidant, and optionally one or more of the following: a taste-masker, a sweetener, a flavoring agent. The fast-dissolving vonoprazan formulation can comprise an oral dosage form or a liquid dosage form. The oral dosage form can comprise a fast-dissolving wafer or an orally disintegrating tablet as described herein.

In another embodiment, the disclosure is to a package comprising the fast-dissolving vonoprazan formulation e.g. produced by the described herein, and comprising vonoprazan or a pharmaceutically acceptable salt thereof, water, a binder, a sugar alcohol, an anti-oxidant, and optionally one or more of the following: a taste-masker, a sweetener, a flavoring agent, wherein the fast-dissolving vonoprazan formulation is provided in unit doses for treatment of gastroesophageal reflux disease (GERD). In one practice, the unit doses are for oral administration, e.g. the unit doses comprise a fast-dissolving wafer or an orally disintegrating tablet. In one practice, the unit doses comprise 1 mg to 50 mg of the fast-dissolving vonoprazan formulation per dose, which 1 mg to 50 mg dose includes all ranges in between, e.g. 2 mg, 3 mg, 4 mg, 5 mg . . . 47 mg, 48 mg, 49 mg.

In another aspect, the disclosure is to a pharmaceutical formulation comprising vonoprazan fumarate and one or more anti-oxidants. In one practice, the anti-oxidant is one or more of the following: ascorbic acid, vitamin E, cysteine, methionine, uric acid, tartaric acid, gallic acid, and sodium metabisulfite. In one particular practice, the pharmaceutical formulation comprises vonoprazan fumarate and one or more of the following anti-oxidants: ascorbic acid, tartaric acid, gallic acid, cysteine.

### Example 4: Fast-Dissolving Vonoprazan Oral Dosage Formulations

Different fast-dissolving vonoprazan oral dosage formulations were prepared each with a different anti-oxidant selected from ascorbic acid, vitamin E/starch blend, vitamin E/TPGS (d-a-tocophenyl polyethylene glycol 1000 succinate), cysteine hydrochloride (HCl), methionine, uric acid, tartaric acid, gallic acid, and sodium metabisulphite. The preparations otherwise comprised water, vonoprazan fumarate, HMW fish gelatin as the binder, mannitol as the alcohol sugar, hydroxypropyl β-cyclodextrin (CyD) (commercially available as Kleptose HPB) as taste-masker when present, sucralose as sweetener when present, and an orange flavored flavoring agent when present.

### Procedure:

The procedure for preparing the nine different fast-dissolving vonoprazan oral dosage formulation was as follows: the binder (e.g. HMW fish gelatin) and sugar alcohol (e.g. mannitol) were added to and mixed in purified water in a stainless-steel vessel while stirring to create a first pre-mix. This first pre-mix was heated at 60° C.±2° C. for 10 minutes or until the gelatin was fully dissolved. The first pre-mix was cooled to 23° C.±2° C. Vonoprazan or a pharmaceutically acceptable salt thereof (e.g. vonoprazan fumarate), taste-masker, when present (e.g. CyD), and the anti-oxidant were mixed in a separate stainless-steel vessel to prepare a second pre-mix. A pipette and spatula were used to gradually wet the second pre-mix with the first pre-mix. Once wetted, the remainder of the first pre-mix was added to form a suspension. The sweetener (e.g. sucralose) and flavoring agent (e.g. an orange flavoring agent), when used, were added to the suspension form a product mix. The product mix was cooled to 23° C.±2° C. with stirring, e.g. via a water bath. The product mix was then dosed into blister packs as follows: Product mix was dosed into blister pockets at a temperature of 10° C.±2° C. The dosed product was then frozen using the following conditions: freezing temp: −70° C.; residence time: 3:15 (min:see). The frozen dosed product was then placed in freezer for a minimum of 24 hours prior to freeze drying using the following conditions: shelf temp: 0° C. drying time: 720 min. The freeze dried product was inspected and then the blisters were sealed using a commercially available packaging machine, e.g. an R750 packaging machine. The sealed dried product was then stored in a dry storage cabinet. The foregoing procedure is non-limiting and variations to this procedure, e.g. sequence of steps, are also contemplated. A lyophilization procedure useful to produce the fast-dissolving vonoprazan oral dosage formulations is the ZYDIS® procedure as known in the art, e.g. as described in U.S. Pat. No. 10,548,839 the contents of which are incorporated herein by reference.

### Formulation A:

An exemplary fast-dissolving vonoprazan oral dosage formulation comprising a taste-masker, sweetener, and flavoring agent were prepared according to the above procedure and had the following formulation:

| Ingredient | Wt % |
|---|---|
| Water | 82.52 |
| HMW fish gelatin | 4.50 |
| Mannitol | 3.60 |
| Vonoprazan Fumarate | 1.48 |
| Kleptose HPB (HP β-CyD) | 6.75 |
| Anti-oxidant | 0.50 |
| Sucralose (micronized) | 0.25 |
| Orange flavor | 0.40 |

In specific practices of Formulation A, the anti-oxidants (at 0.50 wt %) were:
Formulation A1: Cysteine HCl
Formulation A2: Tartaric Acid
Formulation A3: Gallic Acid

### Formulation B:

An exemplary fast-dissolving vonoprazan oral dosage formulation wherein the taste-masker is absent, but the sweetener and flavoring agent are present, were prepared according to the above procedure and had the following formulation:

| Ingredient | Wt % |
|---|---|
| Water | 85.15 |
| HMW fish gelatin | 5.0 |
| Mannitol | 4.0 |
| Vonoprazan Fumarate | 4.45 |
| Tartaric Acid | 0.50 |
| Sucralose (micronised) | 0.50 |
| Orange flavor | 0.40 |

In specific practices of Formulation B, the anti-oxidants (at 0.50 wt %) were:
Formulation B1: Cysteine HCl
Formulation B2: Tartaric Acid
Formulation B3: Gallic Acid

### Formulation C:

Nine exemplary fast-dissolving vonoprazan oral dosage formulations having different anti-oxidants, but not having the taste-masker, the sweetener, and the flavoring agent, were prepared according to the above procedure for Formulation B and had the following anti-oxidants (at 0.50 wt %) formulations:
Formulation C1: Ascorbic Acid
Formulation C2: Vitamin E/starch blend
Formulation C3: Vitamin E/TPGS
Formulation C4: Cysteine HCl
Formulation C5: Methionine
Formulation C6: Uric Acid
Formulation C7: Tartaric Acid
Formulation C8: Gallic Acid
Formulation C9: Sodium Metabisulfite

### NDSRI Levels:

The formulations prepared had NDSRI levels measured twice (in ppm) as herein described at the time the formulation was prepared (SHO) and after 48 hours (SH48). The formulations were exposed to 60° C. conditions for 2 weeks when NDSRI levels were measured again. Results are shown in the Table below (NDSRI level in average ppm of the two measurements):

| Formulation | SH0 | SH48 | 2 Weeks |
|---|---|---|---|
| A1 | 0.37 | 0.47 | 0.47 |
| A2 | 0.89 | 0.77 | 5.10 |
| A3 | 0.65 | 0.54 | 2.37 |
| B1 | 0.32 | 0.33 | 0.28 |
| B2 | 0.37 | 0.40 | 0.37 |
| B3 | 0.40 | 0.42 | 0.47 |
| C1 | 0.38 | N/A | 0.39 |
| C2 | 0.30 | N/A | 2.06 |
| C3 | 0.36 | N/A | 4.66 |
| C4 | 0.24 | N/A | 0.27 |
| C5 | 0.38 | N/A | 5.98 |
| C6 | 0.26 | N/A | 1.08 |
| C7 | 0.26 | N/A | 0.43 |
| C8 | 0.29 | N/A | 0.31 |
| C9 | 0.31 | N/A | 1.60 |

1-79. (canceled)

80. A pharmaceutical composition comprising:
vonoprazan or a pharmaceutically acceptable salt thereof; and
an antioxidant;
wherein N-nitroso-vonoprazan (NDSRI) is absent, or is present in a pharmaceutically acceptable amount, further wherein the pharmaceutically acceptable amount is less than about 2.4 ppm.

81. The composition of claim 80, wherein the antioxidant is selected from ascorbic acid, vitamin E, cysteine, methionine, uric acid, tartaric acid, gallic acid, and sodium metabisulfite.

82. The composition of claim 81, wherein the antioxidant is ascorbic acid.

83. The composition of claim 80, comprising vonoprazan fumarate.

84. The composition of claim 80, wherein the pharmaceutically acceptable amount of N-nitroso-vonoprazan (NDSRI) is less than about 0.60 ppm.

85. The composition of claim 84, wherein the pharmaceutically acceptable amount of N-nitroso-vonoprazan (NDSRI) is less than about 0.20 ppm.

86. The composition of claim 80, wherein the composition has been stored for at least 12 months at 25° C./60% relative humidity (RH).

87. The composition of claim 80, wherein the composition has been stored for at least 12 months at 40° C./75% relative humidity (RH).

88. The composition of claim 82, comprising about 1.4 wt % to about 1.8 wt % of ascorbic acid.

89. The composition of claim 82, comprising up to about 4.0 mg of ascorbic acid.

90. The composition of claim 89, comprising up to about 2.0 mg of ascorbic acid.

91. The composition of claim 80, comprising an effective amount of about 10 mg of vonoprazan.

92. The composition of claim 80, comprising an effective amount of about 20 mg of vonoprazan.

93. The composition of claim 91, comprising from about 13 mg to about 14 mg of vonoprazan fumarate.

94. The composition of claim 92, comprising from about 26 mg to about 27 mg of vonoprazan fumarate.

95. The composition of claim 80, comprising one or more disintegrants, binders, diluents, lubricants, anti-oxidants, colorants, flavoring agents, preservatives or taste-masking agents.

96. The composition of claim 80, wherein the composition is made by a process that includes a wet granulation step.

97. A tablet or capsule comprising:
about 13 mg to about 14 mg of vonoprazan fumarate;
an antioxidant; and
wherein N-nitroso-vonoprazan (NDSRI) is absent, or is present in a pharmaceutically acceptable amount, further wherein the pharmaceutically acceptable amount is less than about 2.4 ppm.

98. The tablet or capsule of claim 97, wherein the antioxidant is selected from ascorbic acid, vitamin E, cysteine, methionine, uric acid, tartaric acid, gallic acid, and sodium metabisulfite.

99. The tablet or capsule of claim 98, wherein the antioxidant is ascorbic acid.

100. The tablet or capsule of claim 97, comprising about 1 mg to about 2 mg ascorbic acid.

101. The tablet or capsule of claim 100, comprising about 1.4 mg to about 1.65 mg ascorbic acid.

102. A tablet or capsule comprising:
about 26 mg to about 27 mg of vonoprazan fumarate;
an antioxidant; and
wherein N-nitroso-vonoprazan (NDSRI) is absent, or is present in a pharmaceutically acceptable amount, further wherein the pharmaceutically acceptable amount is less than about 2.4 ppm.

103. The tablet or capsule of claim 102, wherein the antioxidant is selected from ascorbic acid, vitamin E, cysteine, methionine, uric acid, tartaric acid, gallic acid, and sodium metabisulfite.

104. The tablet or capsule of claim 103, wherein the antioxidant is ascorbic acid.

105. The tablet or capsule of claim 102, comprising about 2.5 mg to about 3.5 mg ascorbic acid.

106. The tablet or capsule of claim 105, comprising about 2.8 mg to about 3.3 mg ascorbic acid.

107. A method of treating gastroesophageal reflux disease (GERD) comprising administering a composition of claim 80.

108. A method of treating a symptom of gastroesophageal reflux disease (GERD) selected from burning sensation in the chest, regurgitation of food or sour liquid, upper abdominal pain, dysphagia, coughing, vocal cord inflammation, or new or worsened asthma comprising administering a composition of claim 80.

109. A method of preparing a pharmaceutical composition comprising:
introducing a mixture of vonoprazan or a pharmaceutically acceptable salt thereof, a filler, and a binder material into a fluidized bed processor;
fluidizing the mixture; and
spraying a composition comprising water, fumaric acid, ascorbic acid, and a binder onto the fluidized mixture.
